(19) United States
(12) Patent Application Publication
(10) Pub. No.: US 2015/0096390 A1
(43) Pub. Date: Apr. 9, 2015

(54) CHANNEL DEVICE, ASSEMBLY MEMBER, METHOD OF FORMING CHANNEL DEVICE, AND METHOD OF INSPECTING CHANNEL DEVICE

(71) Applicant: CANON KABUSHIKI KAISHA, Tokyo (JP)

(72) Inventor: Makoto Ogusu, Yorktown, VA (US)

(21) Appl. No.: 14/488,537

(22) Filed: Sep. 17, 2014

(30) Foreign Application Priority Data
Oct. 7, 2013 (JP) 2013-210195

(51) Int. Cl. B01L 3/00; G01N 33/38; G01B 21/16

(52) U.S. Cl. CPC B01L 3/502707; G01B 21/16; G01N 33/386; B01L 3/502715; B01L 2200/025; B01L 2300/0874; B01L 2200/12; B01L 2300/0816; B01L 2300/0887
USPC 73/865.8; 422/502; 422/503; 29/700; 29/432

(57) ABSTRACT

In order to position components each including a channel in assembly, provided are a positioning method, which is particularly useful in manufacturing a component using an injection molding technology, and a device to which the method is applicable. Specifically, provided is a channel device, including: a first device including a channel; and a second device including a channel, the channel device being formed by joining the first device and the second device to each other so that the channel in the first device and the channel in the second device communicate to each other, the first device having a plurality of holes along an outer side of an edge of a region of the first device, which is joined to the second device.

### BACKGROUND OF THE INVENTION

1. Field of the Invention

The present invention relates to a device configured to exert its function under a state in which two chips are integrated and microchannels of the chips are coupled to each other.

2. Description of the Related Art

In recent years, research and development have been vigorously conducted on a technology called a micro total analysis system (μ-Tas), in which all elements necessary for chemical or biochemical analysis are integrated on one chip. In μ-Tas, such a chip is generally called a microfluidic device, and includes a microchannel, a temperature control mechanism, a concentration adjusting mechanism, a liquid feeding mechanism, a reaction detecting mechanism, and others.

Microfluidic devices have been vigorously developed in recent years. Among others, a DNA analysis device aiming at examination to obtain genetic information such as a single nucleotide polymorphism (SNP) of a human genome is particularly attracting attention, and research thereof is vigorously conducted.

DNA analysis involves the following two steps: (1) a step of amplifying DNA; and (2) a step of determining the DNA.

Polymerase chain reaction (PCR) is generally used in the step (1) of amplifying DNA. This is a method of amplifying DNA by mixing a primer complementary to a part of the DNA to be amplified and an enzyme or the like with the DNA to be amplified and subjecting the mixture to a thermal cycle. This step requires accurate and high speed temperature control for the purpose of shortening reaction time.

There are many ways to perform the step (2) of determining the DNA. For example, a thermal melting method may be used in determining a SNP. The thermal melting method is a method of detecting a melting temperature (hereinafter referred to as Tm) of DNA by gradually raising a temperature of a DNA solution after PCR. When the temperature is low, DNA intercalated with a fluorochrome forms a double strand, and thus, a fluorescent signal is detected. After that, the temperature gradually rises, and, when the temperature reaches Tm, the double-stranded DNA is separated into single strands, and thus, the intensity of the fluorescent signal is abruptly lowered. Tm is determined based on this relationship between the temperature and the fluorescent signal, to thereby detect the SNP. In this step, the DNA is determined by comparing values of Tm, and thus, accurate temperature measurement is required.

As described above, when DNA is analyzed, temperature control is important, and in particular, high speed and accuracy are required for the temperature control. In order to realize high speed, it is advantageous that a solution to react have a small volume. Therefore, a method of holding a solution in a microchannel is favorably used. Further, in order to cause reaction to occur with stability, a channel is in some cases formed in a glass chip so as to measure the reaction using a fluorochrome or the like by utilizing a high transmittance thereof. Further, in order to enhance processing ability of the entire device, the device includes a plurality of channels in some cases. The channels are densely arranged. Microchannels are required to be magnified when observed, and dense arrangement thereof enables collective obtainment of information about the plurality of channels along with magnification thereof. While the inside of the microchannel is a microsystem, a system provided up to a process in which a reagent is introduced into a reaction chip is a macrosystem. In particular, portions at which a reagent is introduced into the respective channels are required to have a predetermined size. In view of the required size in a range of from portions at which the reaction occurs in the end to the portions at which a reagent is introduced, these reaction chips have microstructures, and are required to have a predetermined size. Therefore, it is desired that a reaction field that requires a microstructure be formed as a glass chip, and that, taking costwise advantage into consideration, the function of converting the size from a microsystem to a macrosystem be performed using a microchannel formed of plastic. It is desired that the microchannel formed of plastic be formed by injection molding from the viewpoint of smoothness of an inner surface of the channel and cost.

FIG. 9 is a top view of a related-art chip, and FIG. 10 is a side view of the related-art chip. A plastic chip 110 includes a glass chip 101 at a center thereof. The plastic chip 110 includes microchannels 104, which are coupled to microchannels 102 in the glass chip 101 at coupling portions 103, respectively. The microchannels 102 are heated by a heater (not shown) to cause various kinds of chemical reaction to occur therein. Liquid pools called wells 105 and 106 are formed at ends of the microchannels 104, respectively, in the plastic chip 110. A pipet robot 107 illustrated in FIG. 10, which has the same function of a pipet, supplies a reagent to the wells 105 serving as inlets. The reagent, which passes through the microchannels, accumulates in the wells 106 formed similarly at opposite ends of the microchannels. Holds 108 are connected to the wells 106 on an outlet side. Due to negative pressure applied through pressure transfer tubes 109, the reagent is drawn into the microchannels and the reagent is stopped at desired positions, respectively.

In the related art described above, at the portions 103, which couple the microchannels in the plastic chip 110 to the microchannels in the glass chip 101, in order to allow an error in manufacture and assembly, the diameters of the microchannels in the plastic chip 110 and of the microchannels in the glass chip 101 are set different from each other. However, the difference in diameter is required to be small to the extent that the reagent does not dwell at the level difference at the coupling portions 103, and a high level of accuracy is required in positional adjustment in assembly. Hitherto, a complicated step of performing positioning with a positional adjustment jig while visually observing the coupling portions that are magnified, and then feeding an adhesive for fixation is necessary. Therefore, in order to shorten a tact time for production, there are required measures for positioning components by a simple positioning method instead of the complicated assembly step.

In view of the above-mentioned requirement, a step of positioning through provision of a protrusion on the plastic chip by a pressing method may be conceived. However, when a local protrusion having a height equivalent to a thickness of an entire component is provided, a surface on an opposite side to the protrusion is liable to have a recessed shape due to contraction in the molding step. In the plastic chip, the surface on the opposite side to the protrusion is bonded to form microchannels, and thus, when the flatness of the surface is unsatisfactory, a poorly bonded portion is generated between the microchannels, which may be a liquid pool or may cause a microchannel to be connected to an adjacent microchannel. Further, a side surface of the protrusion is a positioning reference, but, in order to release the plastic chip from a mold, the side surface of the protrusion is required to be slanted. When a component is fitted on the protrusion for positioning, the fitted component is liable to override the slanted side surface to cause an error.

Alternatively, a method of performing positioning with an outer peripheral edge portion of the plastic chip set as a reference position may be conceived. However, generally, a distance between the reference position and a coupling hole to be positioned is large. Therefore, in view of fluctuations in contraction in molding, it is difficult to mold the plastic chip with an accurate positional relationship between the outer peripheral edge portion and the coupling hole. As described above, it is a challenge to establish, instead of a visual positioning method, a method of performing simple positioning with high accuracy under a state in which the liquid pool is as small as possible, and to establish a device to which the method is applicable.

### SUMMARY OF THE INVENTION

The present invention relates to a channel device, including: a first device including a channel; and a second device including a channel, the channel device being formed by joining the first device and the second device to each other so that the channel in the first device and the channel in the second device communicate to each other, the first device having a plurality of holes along an outer side of an edge of a region of the first device, which is joined to the second device.

In the channel device according to one embodiment of the present invention, the devices that form the channel device can be positioned simply and with high accuracy.

Further features of the present invention will become apparent from the following description of exemplary embodiments with reference to the attached drawings.

### BRIEF DESCRIPTION OF THE DRAWINGS

FIG. 1 illustrates a channel device according to a first example of the present invention.

FIG. 2 illustrates a positioning tool to be used in the first example of the present invention.

FIG. 3 is a sectional view illustrating positioning in the first example of the present invention.

FIG. 4 is a sectional view illustrating positioning in a second example of the present invention.

FIG. 5 is a sectional view illustrating positioning in a third example of the present invention.

FIG. 6 illustrates a channel device according to a fourth example of the present invention.

FIG. 7 is a sectional view illustrating positioning in the fourth example of the present invention.

FIG. 8 is a sectional view illustrating the positioning in the fourth example of the present invention.

FIG. 9 is a top view of a related-art microfluidic device.

FIG. 10 is a sectional view of the related-art microfluidic device.

### DESCRIPTION OF THE EMBODIMENTS

The present invention is described in detail below.

The present invention is described in more detail by way of the following examples.

### First Example

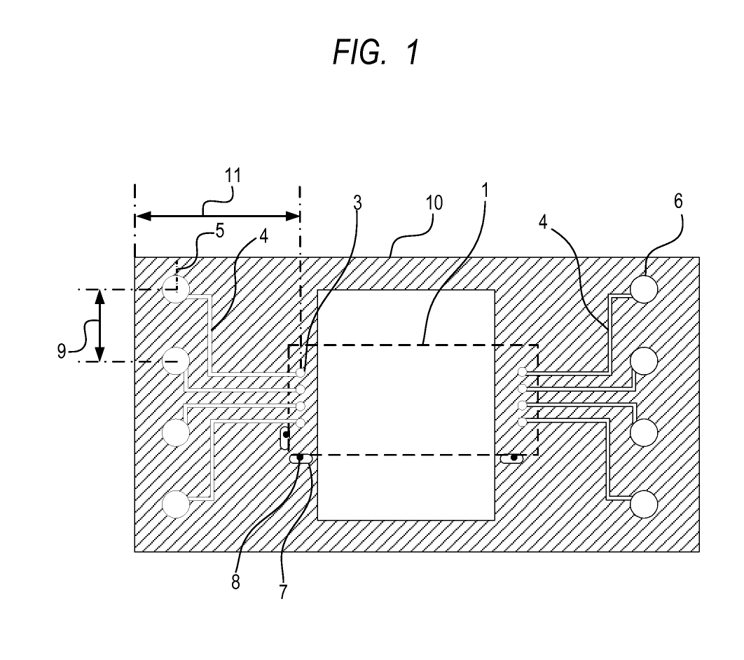

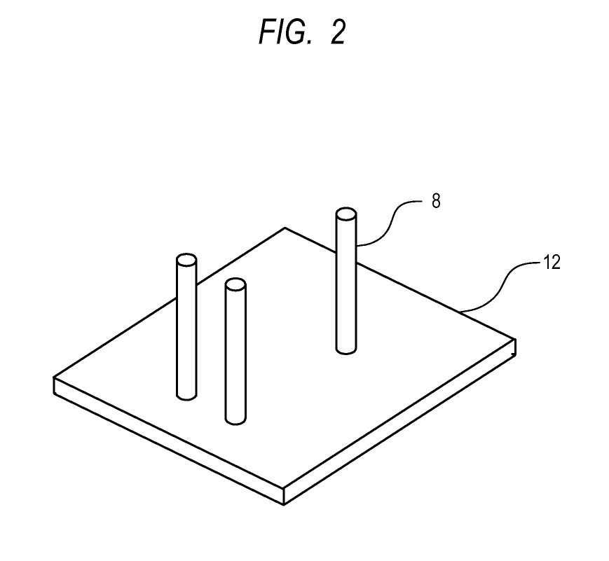

FIG. 1 illustrates a channel device according to a first example of the present invention. With reference to FIG. 1, a flat plate-like plastic chip 10 serving as a first device includes inlet-side wells 5, outlet-side wells 6, coupling portions 3, and microchannels 4 for connecting the inlet-side wells 5 and the coupling portions 3 and for connecting the outlet-side wells 6 and the coupling portions 3, respectively. A flat plate-like chip formed of glass (glass chip 2) serving as a second device and including microchannels is assembled at a position 1 so that the channels 4 in the plastic chip 10 communicate to the channels in the glass chip 2, using a tool (assembly member) 12 illustrated in FIG. 2. The plastic chip 10 further includes a plurality of through holes 7 serving as positioning references with the glass chip 2. The holes 7 are formed along an outer side of edges of a region in which the plastic chip 10 and the glass chip 2 are joined to each other. The holes 7 may penetrate the plastic chip 10. The tool 12 is a positioning tool, and includes a plurality of pins (protrusions) 8. The pins 8 are provided at positions facing the holes 7, respectively, and are, in assembly, inserted into the holes 7 to be in abutment against side surfaces of the holes 7 on the mounted glass chip sides, respectively. In this state, the glass chip is pressed against the pins 8, and thus the glass chip is positioned at the desired glass chip mounting position 1. It is therefore preferred that three or more holes 7 be formed in the plastic chip 10 and at least one of the holes 7 be formed at a position, which is out of a straight line connecting other two of the holes 7. Further, it is preferred that three or more pins 8 be provided on the tool 12 and at least one of the pins 8 be provided at a position, which is out of a straight line connecting other two of the pins 8. The pins 8 have a sectional shape that can be inserted into the holes 7, and the shape is not specifically limited insofar as the pins 8 have a length, which is equal to or larger than a thickness of the glass chip 2.

Note that, although not illustrated in FIG. 1, in this example, the plastic chip 10 is formed by bonding two members. In this case, by forming grooves in any one of the members, the channels 4 are formed by the bonding.

When liquid is retained at the coupling portion 3 that is nearer to the inlet-side well 5, there may occur contamination in inspections that are performed continuously, and thus, an extreme level difference at side surfaces of the channels and extreme change in cross sectional area of the coupling portion are not desired. Therefore, it is preferred that the coupling portions 3 that are nearer to the inlet-side wells 5 be designed to have a hole diameter that is as close to the size of the microchannels as possible and to have a small diameter difference between the plastic chip 10 and the glass chip 2. On the other hand, at the coupling portions 3 that are nearer to the outlet-side wells 6 after various kinds of inspections in the glass chip, it is not necessary to be so nervous about dwelling of liquid and the like, and thus, the coupling portions 3 that are nearer to the outlet-side wells 6 are tolerant even when the hole diameter at one of the plastic chip 10 and the glass chip 2 is set larger than that at the other of the plastic chip 10 and the glass chip 2, and desired assembly accuracy may be designed to be relaxed. As a result of placing importance on the coupling portions 3 that are nearer to the inlet-side wells 5 in this way, in FIG. 1, two out of the three positions at which the pins 8 are pressed are arranged in proximity to the coupling portions 3 that are nearer to the inlet-side wells 5.

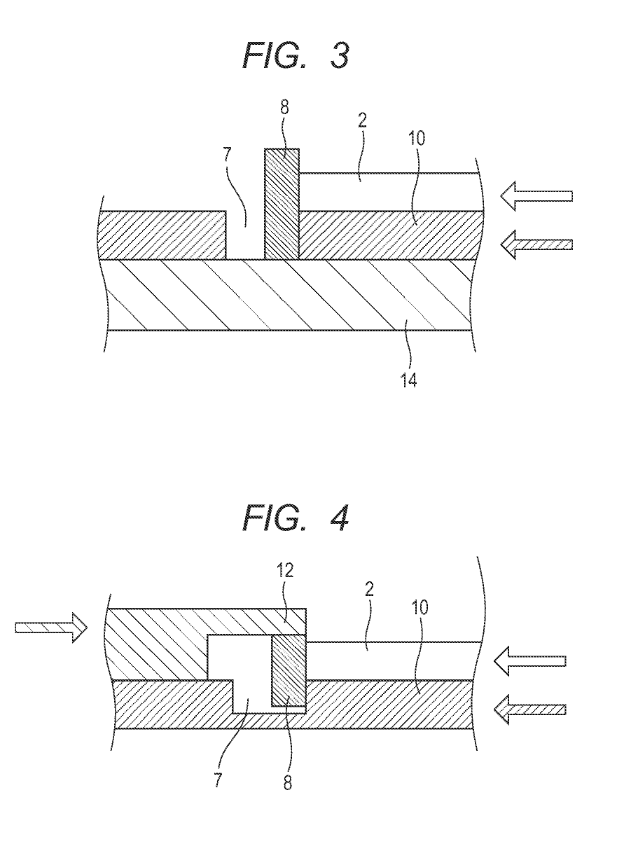

FIG. 3 is a sectional view taken through a hole 7 and a pin 8 when the glass chip 2 is assembled in the plastic chip 10. Both the plastic chip 10 and the glass chip 2 are pressed against the tool 12 including the pin 8. In this example, the plastic chip 10 and the glass chip 2 are pressed against the pins 8 at least at three positions, which are not in a straight line, and thus, satisfactory positioning can be performed.

In this example, the positioning is performed in a device including four microchannels. Note that, in the present invention, the microchannel means a channel having a channel diameter of 1 mm or less, and a device including channels having such a channel diameter is referred to as a microchannel device. At a portion for introducing a reagent into the microchannel, a pipet robot has a given size, and thus, an interval 9 between the inlet-side wells 5 is 10 mm. An interval between the microchannels in the glass chip 2 is about 0.5 mm, and thus, the interval between the channels becomes an order of magnitude greater. Further, a measurement system (not shown) such as an optical measurement device for obtaining a result of inspection in the glass chip 2 is necessary. An interval 11 between an end of a contour of the plastic chip 10 and the coupling portion 3 is several tens of millimeters so that the above-mentioned pipet robot and the like do not obstruct operation of the measurement system. In this example, acrylic is used as a material of the plastic chip 10 and a glass chip 2, but the material of the plastic chip 10 and the glass chip 2 is not specifically limited. Note that, using a transparent material as the glass chip 2 enables optical observation of an inside of the microchannels, and also enables measurement of reaction using a fluorochrome or the like and collective obtainment of information about the plurality of channels along with magnification thereof. Further, in the plastic chip 10, the size may be converted from a microsystem to a macrosystem, and thus, it is desired that, taking costwise advantage into consideration, a plastic material be used. Through formation of the plastic chip 10 by injection molding, smoothness of an inner surface of the channels can be secured and the cost can be reduced.

The coupling portion 3 has a hole diameter difference of 0.1 mm between the plastic chip 10 and the glass chip 2, and thus, a tolerance of +/−30 μm is set for the respective holes. Acrylic has a linear expansion coefficient of 6×10⁻⁵[1/° C.]. Assuming that a temperature in the molding is about 90° C., in order to cause change in length due to contraction between the temperature at the molding and room temperature (assumed to be 23° C. in this case) to be 30 μm or less, the positioning reference (hole 7) and the target to be positioned (coupling portion 3) are required to be in proximity to each other with an interval therebetween set to about 7.4 mm or less, or to 10 mm or less even when the size of the coupling portion is taken into consideration. The microchannel is formed near the coupling portion, and thus, when a protrusion is formed, it is difficult to place the protrusion in proximity to the target. On the other hand, in this example, the positioning reference is a hole, and thus, can be placed in proximity to the target without greatly deteriorating the flatness. Further, the plastic chip 10 has a substantially uniform thickness, and thus, satisfactory flatness of the component can be secured even when the plastic chip 10 is then bonded to a substrate to form the channels 4. As a result, it is assumed that no contamination of liquid occur between adjacent channels.

### Second Example

FIG. 4 illustrates a second example of the present invention. While the holes 7 are through holes in the first example, holes 7, which are blind holes, are adopted in the second example. Under conditions similar to those of the first example except for this condition, the tool 12 used for the positioning is placed from the mounted glass chip side as illustrated in FIG. 4, and the pin 8 is pressed against a side surface of an opening of the positioning hole. By pressing the glass chip 2 against a side surface of the tool, satisfactory positioning can be performed.

### Third Example

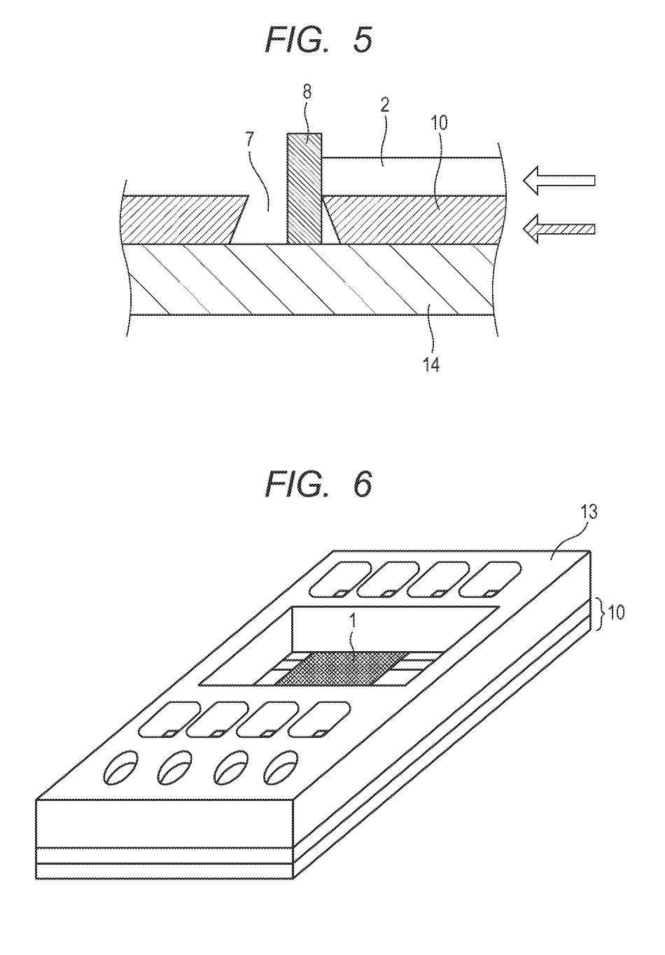

FIG. 5 illustrates a third example of the present invention. In the third example, similarly to the first example, the through holes 7 are used as positioning references. In this case, in order to readily release the molded component from a mold 14, the hole 7 is tapered so that a width thereof increases toward the mold 14 (as being away from a surface in contact with the glass chip 2). Except for this condition, the second example is similar to the first example. In this example, by tapering the holes 7, the releasability is satisfactory. In addition, the pin 8 is in contact with a portion of the hole 7, at which an inner diameter thereof is the smallest, and thus, satisfactory positioning can be performed.

### Fourth Example

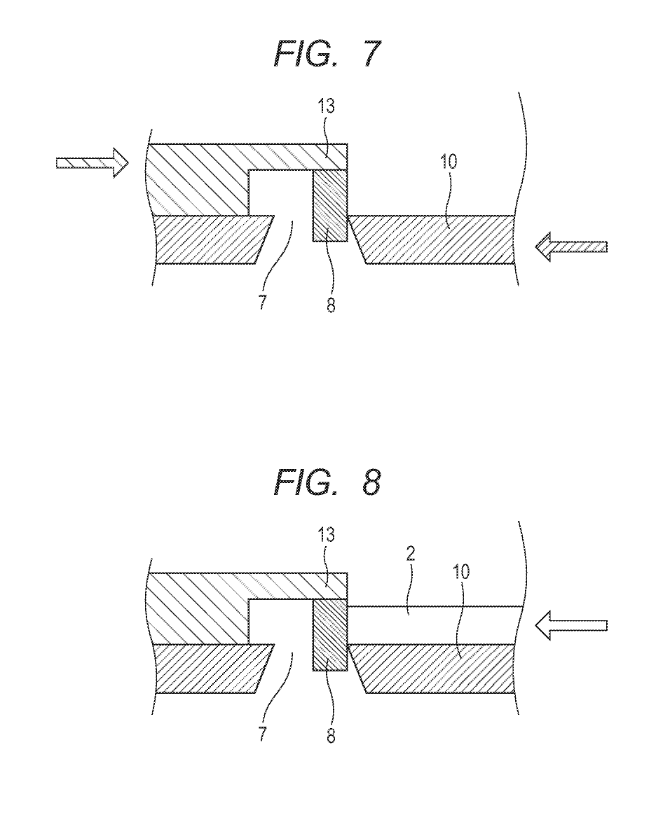

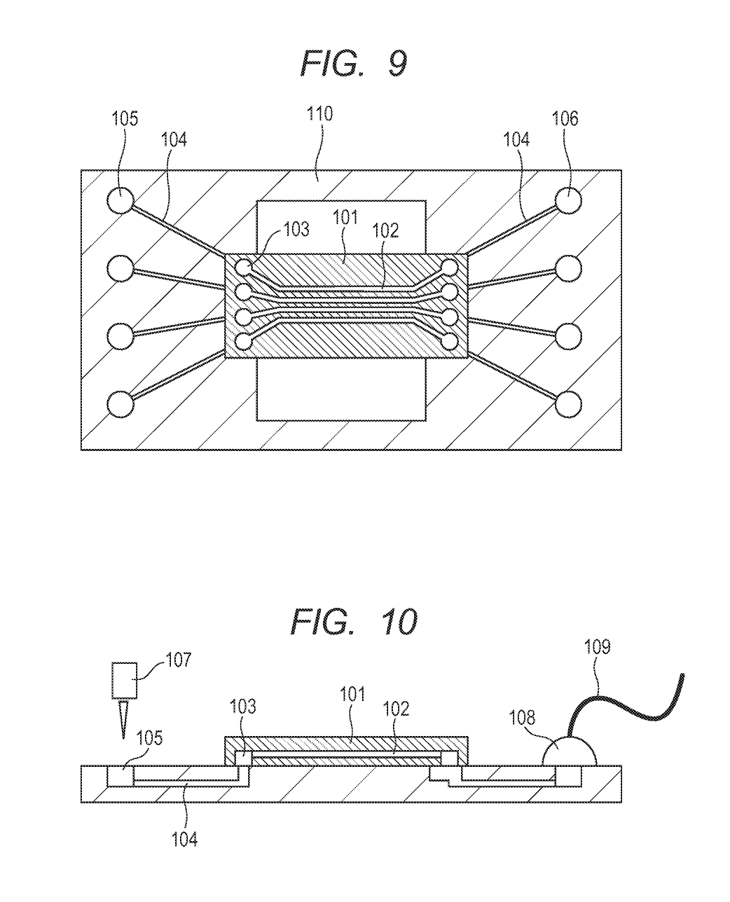

FIGS. 6 to 8 illustrate a fourth example of the present invention. In this example, the device includes a well plate 13 in addition to the plastic chip 10 having the microchannels formed therein. The well plate 13 increases a thickness of the component. By mounting the well plate 13 on the plastic chip 10 having the microchannels formed therein, a capacity of the wells is increased. Further, in this case, other wells for containing a reagent to be used for inspection are formed. Although the well plate 13 is thick and complicated in shape including a large window for observing the glass chip, various wells, and the like, the well plate 13 does not form microchannels by being bonded to the plastic chip 10, and thus, is tolerant about flatness and positioning thereof. In this example, a protrusion 8 that functions similarly to the pin 8 is provided to the well plate 13. The protrusion 8 may have the shape of a pin similarly to the first to third examples, but has, in this example, a rectangular parallelepiped shape with a surface on a side to be joined to the glass chip 2. Reference is now made to FIGS. 7 and 8 to describe this example in further detail. FIGS. 7 and 8 are sectional views illustrating a portion against which the glass chip is pressed to perform the positioning. In FIG. 7, the well plate 13 includes the protrusion 8, which can be inserted into the hole 7 in the plastic chip 10. The protrusion 8 has a continuous side surface extending to a position at which the glass chip 2 can be brought into contact therewith. As illustrated in FIG. 7, first, this protrusion 8 is used to press the well plate 13 against the plastic chip 10 and bonding is performed while the positioning is performed. Then, as illustrated in FIG. 8, the glass chip 2 is pressed against the side surface of the protrusion 8 for positioning. In this way, satisfactory positioning can be performed.

Note that, the present invention is described above taking a microchannel device as an example, but the present invention is not limited thereto, and may be applied to a channel device including a channel having a channel diameter of 1 mm or more.

As described in the examples above, it is preferred that both the plastic chip 10 and the glass chip be formed of a flat plate-like member having a flat surface that can be used for joining the plastic chip 10 and the glass chip 2 together, and that the plastic chip 10 and the glass chip 2 be stacked and joined together to form a channel device.

Further, it is preferred that the plastic chip 10 have a hollow structure so that, when the glass chip 2 is stacked thereon, observation of a region to be observed (not shown) in the glass chip 2 is not hindered. The hollow structure is illustrated as a white rectangle in FIG. 1.

Further, the positional relationship between the holes and the side surface (end) of the glass chip described in the above-mentioned examples may be used for determining whether the joined state is satisfactory or not. Specifically, the positional relationship may be used for determining whether satisfactory positioning has been performed or not in the channel device manufactured through the joining.

While the present invention has been described with reference to exemplary examples, it is to be understood that the invention is not limited to the disclosed exemplary examples. The scope of the following claims is to be accorded the broadest interpretation so as to encompass all such modifications and equivalent structures and functions.

This application claims the benefit of Japanese Patent Application No. 2013-210195, filed Oct. 7, 2013, which is hereby incorporated by reference herein in its entirety.

What is claimed is:

1. A channel device, comprising:
a first device comprising a channel; and
a second device comprising a channel,
the channel device being formed by joining the first device and the second device to each other so that the channel in the first device and the channel in the second device communicate to each other,
the first device having a plurality of holes along an outer side of an edge of a region of the first device, which is joined to the second device.

2. A channel device according to claim 1,
wherein the first device has three holes or more, and
wherein at least one of the plurality of holes is formed at a position, which is out of a straight line connecting other two of the plurality of holes.

3. A channel device according to claim 1, wherein the second device is formed of a transparent material.

4. A channel device according to claim 1, wherein at least one of the plurality of holes penetrates the first device.

5. A channel device according to claim 1, wherein the plurality of holes are tapered.

6. A channel device according to claim 1, further comprising a third device,
wherein the third device comprises a protrusion insertable into each of the plurality of holes, and
wherein the protrusion has a length equal to or larger than a thickness of the second device.

7. A channel device according to claim 1,
wherein the first device is formed by joining two members, and
wherein the channel in the first device is formed by forming a groove in any one of the two members and joining the two members.

8. A channel device according to claim 1, wherein each of the first device and the second device comprises a flat plate-like member having a flat surface.

9. A channel device according to claim 8, wherein the first device and the second device are joined to each other so that the flat surface of the first device and the flat surface of the second device are opposed to each other and are overlaid on each other.

10. A channel device according to claim 8, wherein the first device has a hollow structure.

11. An assembly member to be used when assembling a channel device comprising a first device comprising a channel and a second device comprising a channel, the channel device being formed by joining the first device and the second device to each other so that the channel in the first device and the channel in the second device communicate to each other,
the first device comprising a plurality of protrusions along an outer side of an edge of a region of the first device, which is joined to the second device,
the plurality of protrusions having a thickness equal to or larger than a thickness of the second device.

12. A method of forming a channel device comprising a first device comprising a channel and a second device comprising a channel, the channel device being formed by joining the first device and the second device to each other so that the channel in the first device and the channel in the second device communicate to each other, the method comprising:
forming a plurality of holes along an outer side of an edge of a region of the first device, which is joined to the second device;
inserting, using an assembly member comprising a protrusion having a length equal to or larger than a thickness of the second device, the protrusion of the assembly member into each of the plurality of holes in the first device; and
positioning the first device and the second device with respect to each other.

13. A method of inspecting a channel device comprising a first device comprising a channel and a second device comprising a channel, the channel device being formed by joining the first device and the second device to each other so that the channel in the first device and the channel in the second device communicate to each other,
the first device of the channel device having a plurality of holes along an outer side of an edge of a region of the first device, which is joined to the second device,
the method comprising inspecting a positional relationship between the plurality of holes and an end of the second device.
